(19) United States
(12) Patent Application Publication
(10) Pub. No.: US 2022/0342242 A1
(43) Pub. Date: Oct. 27, 2022

(54) EXTRACORPORAL BLOOD TREATMENT MACHINE COMPRISING A VIEWING WINDOW WITH ELECTRICALLY SWITCHABLE FILM

(71) Applicant: B. Braun Avitum AG, Melsungen (DE)

(72) Inventor: Waldemar Janik, Melsungen (DE)

(21) Appl. No.: 17/642,416

(22) PCT Filed: Sep. 17, 2020
(86) PCT No.: PCT/EP2020/076020

§ 371 Date: Mar. 11, 2022

(30) Foreign Application Priority Data
Sep. 18, 2019 (DE) 10 2019 125 174.5

(51) Int. Cl. G02F 1/137; A61M 1/36; G02F 1/133

(52) U.S. Cl. CPC G02F 1/137; A61M 1/367; G02F 1/13306; A61M 2205/583

(57) ABSTRACT

A medical device for extracorporeal blood treatment having a housing section that is designed such that is can reversibly change between two transparency states by applying an electrical voltage.

### CROSS-REFERENCES TO RELATED APPLICATIONS

This application is the United States national stage of International Application No. PCT/EP2020/076020, filed Sep. 17, 2020, and claims priority to German Application No. 10 2019 125 174.5, filed Sep. 18, 2019. The contents of International Application No. PCT/EP2020/076020 and German Application No. 10 2019 125 174.5 are incorporated by reference herein in their entireties.

### FIELD

The invention relates to a medical device for extracorporeal blood treatment having a switchable housing section.

### BACKGROUND

A dialysis device/dialysis machine enables patient-specific removal of solutes/dissolved substances (e.g., urea, creatinine, vitamin B12 or β2-microglobulin) and, if applicable, a defined water content from the blood during renal replacement treatments. Dialysis devices are used for both hemodialysis and hemodiafiltration. Basically, dialysis machines can be divided into the following modules: extracorporeal blood circuit, dialysis fluid circuit, disinfection unit, control unit and power supply unit. In addition, consumables, so-called disposables, are used during treatment. These disposables include, for example, cannulas, blood hose systems, dialyzers, dialysis concentrates, etc.

Medical devices for extracorporeal blood treatment are connectable to the blood circuit of a patient via extracorporeal lines. Furthermore, the medical device for extracorporeal blood treatment includes a blood pump, a control unit for controlling the blood pump and for monitoring operating conditions. A typical treatment performed by such devices is the dialysis treatment, which is usually performed in special buildings called dialysis centers. Usually, 20 to 50 treatment stations are provided, spread over several rooms. Nursing staff is responsible for monitoring patients during treatment. Efforts are therefore being made to transfer at least part of the monitoring of the patient to the medical device, and in doing so to recognize dangers to the patient, to perform appropriate safety controls, and to call the nursing staff to the patient.

In extracorporeal blood treatment, for example hemodialysis or plasma treatment, a patient's blood flows from an arterial vascular access via a filter to a venous vascular access. An arterial-venous fistula is often surgically created as an access to the vascular system, generally punctured with an arterial and venous cannula (double needle). Likewise, the use of a vascular implant (shunt) is possible. A vascular access is defined as any type of access to the patient's vascular system, but in particular a connection between the patient's artery and vein.

Approaches for controlling a medical device in extracorporeal blood treatment are known from the prior art. For example, US 2015/0253860 A1 describes a control of a dialysis machine using an electric field, wherein the machine can be controlled by including gestures of a user/patient. EP 2 857 053 B1 similarly describes detection of gestures in the detection area of a motion detection device in order to control a dialysis machine, for example using a camera and a cameraless sensor. DE 10 2006 060 819 A1 describes a dialysis machine in which information about a patient's breathing rate is further used for dialysis treatment.

Furthermore, numerous different methods and devices for general monitoring of vital parameters of patients are known. For example, US 2007/0118054 A1 discloses a method and system for monitoring vital parameters, wherein, for example, recording of breathing is made to detect different breathing patterns. A sensor is placed below the patient and can then detect corresponding breathing patterns. These serve to monitor vital functions to predict and treat physiological conditions such as asthma, hypoglycemia, cough, edema, and sleep apnea. US 2008/0269589 A1 describes a portable radar sensor that transmits a radar signal into the chest and receives the reflected signal (back). The device is used to measure and monitor the mechanical cardiac activity of a patient. U.S. Pat. No. 4,958,638 A describes a radar technology that can measure a patient's heart and respiratory rate at a range of approximately 6 meters. The contactless vital sign monitor is used in support of therapies in hospitals as well as in nursing homes. U.S. Pat. No. 3,483,860 A describes detection of respiratory rates of a patient without radar technology, wherein a transmitter sensor is placed above and a receiver sensor below the patient.

In addition, approaches for monitoring a patient during extracorporeal blood treatment are known. A general approach is disclosed in EP 1 574 178 A1, specifically in the form of a medical treatment system in which a video camera is directed at a treatment station. The image from the video camera is displayed on the screen of a remotely located physician's station. In this way, the physician can visually see or respectively monitor the patient. However, it is a disadvantage that continuous monitoring is not possible and only a few patients can be monitored at a time.

The disadvantage of all the aforementioned solutions is that only checking of the patient or checking in the patient's area takes place. Given that the patient is fully monitored by the aforementioned solutions, the patient is thus part of a process which is a necessary measure for him/her, but which is perceived as unpleasant if he/she is constantly monitored. Furthermore, according to experience reports, for a large number of patients it is not a positive experience to see their own blood circulating in a machine. For nursing staff and physicians, on the other hand, it is essential to view the individual components of the medical device in order to be able to check and ensure that the treatment is functioning and proceeding correctly, even in the event that one of the safety devices described above has sounded an alarm.

### SUMMARY

Based on the prior art described above, the object of the present invention is to eliminate the disadvantages mentioned above, in particular to design a medical device with an extracorporeal blood circuit in such a way that medical personnel can view the components or modules, while these remain hidden from a patient.

A medical device according to the invention for extracorporeal blood treatment comprises an extracorporeal blood hose system and a housing. Of course, the medical device according to the invention is not limited to these modules, but may also comprise a dialysis fluid circuit, a disinfection unit, a control unit and power supply as well as a blood pump for the extracorporeal blood circuit, a control unit for controlling the blood pump and for monitoring operating states. The housing consists of a plurality of housing sections, wherein at least one of these housing sections preferably has a multi-part structure. According to the invention, a part of this housing section is connected to an electrical voltage source and has a visual transparency that is reversibly changeable as a function of the currently applied electrical voltage. At least two (voltage/transparency) states of the part can be implemented.

This configuration has the advantage that blood-carrying lines can be made visually visible and invisible within the housing without having to open the housing or having to mechanically move additional components. This means that smooth/edgeless housing sections can be maintained, which is particularly important in terms of hygiene.

In a preferred embodiment, the housing part which can be subjected to voltage is non-transparent in a first state, also referred to as initial state. In particular, when the part of the housing section is not subjected to electrical voltage, the entire housing section is opaque/non-transparent.

In this configuration example, an interior of the housing is not visible, at least through this housing section. In other words, the housing section in the first state is a visual cover that hides the interior of the medical device from outside view.

In a further configuration example, the housing part is transparent in a second state, also referred to as the final state. In particular, when the part of the housing section is under electrical voltage, i.e., an electric circuit is being/is connected to the voltage source, the entire housing section is largely transparent. In the present case, largely transparent means that one hundred percent transparency is almost impossible to implement, regardless of the choice of material of the housing section and its parts. In the present application, transparent means a maximum transparency that can be achieved by technical means and that is perceived by the human eye (as transparent).

According to this configuration example, the interior of the housing is visible through the housing section. Medical personnel, nursing staff, technical staff or other authorized and trained persons thus have the possibility to view the interior of the medical device at one glance and to carry out a visual inspection of the modules inside.

It is particularly preferred if it is possible to switch back and forth between the two states, i.e. the first and the second state. The switching itself can be performed via a conventional switch, a remote control, a touchpad, or otherwise directly on the medical device. A kind of remote access, in the case of a fully networked medical device via the control personnel in a dialysis center, would also be conceivable. In addition to switchability, i.e. ‘on-off’, also dimmability between the initial state and the final state is basically conceivable.

It has proven particularly advantageous if the part of the housing section responsible for transparency/non-transparency corresponding to the two states is or has a switchable foil. This foil is arranged between two basically transparent layers. These other layers may be made, for example, of glass or acrylic glass or any other material that meets the requirements for a certain transparency and a disinfectant resistance. As already mentioned, the foil is preferably non-transparent in its initial state. When an electrical voltage is applied to it, i.e. a switching operation is performed, the foil becomes transparent.

It has proven particularly advantageous if the foil consists of a polymer liquid crystal film. Alternatively, nano-crystallines or so-called micro-blinds may also be used. The foil may also be electrochromic in principle or in the form of a kind of suspended-particle device. The basic requirement is that by applying an electrical voltage to the foil, a structural change occurs within the foil which alters the transmission of the incident light according to the initial and final state described above.

According to the invention, a housing section is usable for a medical device as described above.

A method according to the invention provides that an electrical voltage is applied to an initially non-transparent part of a housing section of an extracorporeal blood treatment machine as described above, thereby changing the transparency of the housing section to a transparent housing section. This can happen immediately (abruptly) or in the form of a stepless or stepped (gradual) dimmability.

A treatment station according to the invention with a treatment couch and a medical device described above is arranged on a wall and has switchable separation walls to separate it from neighboring treatment stations. These separation walls are based/oriented in their mode of operation on/towards the housing sections of the medical device and serve to ensure the privacy of the patients as soon as they are placed in a non-transparent state after the start of treatment.

### BRIEF DESCRIPTION OF THE DRAWING FIGURES

Preferred configuration examples of the invention are explained in more detail below with reference to schematic drawings. The following is shown:

FIG. 1 shows a three-dimensional schematic illustration of a medical device according to the invention;

FIG. 2 shows a schematic layered structure of a housing section;

FIG. 3 shows a medical device with a housing section switched to transparent;

FIG. 4 shows another configuration example of a medical device;

FIG. 5 shows another configuration example of a medical device; and

FIG. 6 shows a treatment station with a medical device according to the invention.

### DETAILED DESCRIPTION

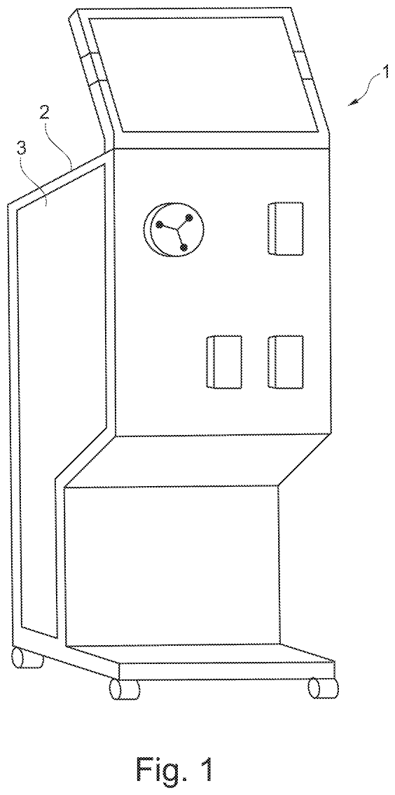

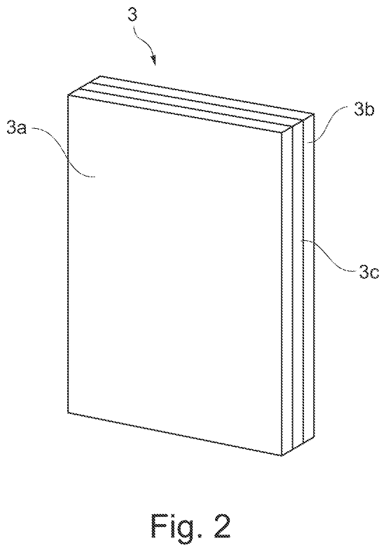

FIG. 1 shows a medical device according to the invention for extracorporeal blood treatment, in this case a dialysis machine 1, with a housing 2 and an electrically switchable housing section 3, which is part of the housing 2. The housing section 3 can preferably assume two (transparency) states, namely an initial state, here the housing section 3 is non-transparent or opaque, and a final state, in which the housing section 3 is transparent. Final state in this context does not mean that this state is permanently maintained after switching. Rather, it is possible to switch back and forth between the initial state and the final state as often as desired. The states from an opaque to a transparent housing section 3 are therefore to be regarded as reversible. In further embodiments, intermediate states of different partial transparency are also conceivable, so that the housing section 3 may also be dimmed continuously or in steps. The housing section may change its state as soon as an electrical voltage is applied. In the voltage-free state, the housing section 3 is non-transparent. In particular, the housing section 3 may be designed as a pane with multiple layers which is inserted into a housing opening. A detailed structure of the pane is shown in FIG. 2.

FIG. 2 shows a three-layer structure of such a pane of the housing section 3 of a medical device according to the invention. The layers 3a and 3b are made of a basically transparent material. This material is, for example, glass or acrylic glass. Other transparent materials can of course also be used, as long as they are resistant to disinfection, since the area of application is a medical device. Between the two layers 3a and 3b, which can be regarded as the outer and inner layer, there is an electrically switchable/actuable foil 3c, as the middle layer, so to speak. In the present embodiment, this foil 3c is designed as a polymer liquid crystal film whose conductive surfaces are connected to an electrical voltage source. The connection is not shown in FIG. 2, since only the schematic structure of the housing section 3 is to be shown. A contacting of a switchable foil 3c is sufficiently known to a person skilled in the art. When an electrical voltage is applied to the foil 3c of the housing section 3, which is in the initial state, an electrical field causes the liquid crystals to be oriented, allowing light to pass through the pane of the housing section 3 almost undisturbed. The pane of the housing section 3 may be rigid or flexible depending on the materials of the layers 3a and 3b.

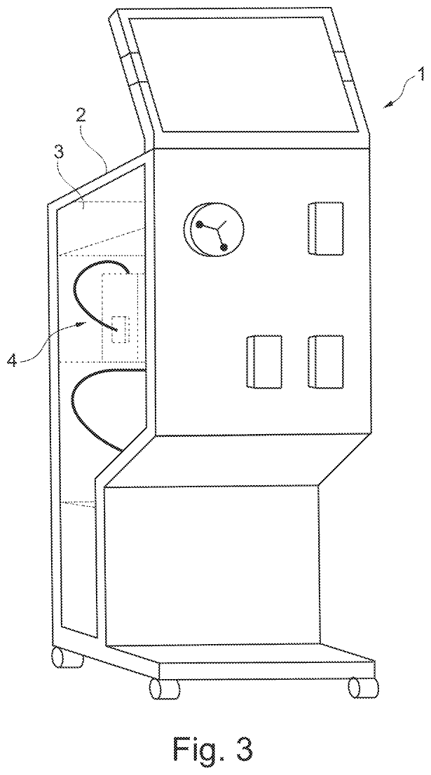

FIG. 3 shows the medical device 1 according to the invention with a pane that can be supplied with current. The housing 2 has the housing section 3, which is switched to transparent, on the left-hand side in this illustration. This means that an electrical voltage is applied to the foil 3c, which is not explicitly shown here, so that the liquid crystals contained therein are formed in the direction of maximum transparency, as already explained above. As soon as the voltage is applied, it is possible for an observer, in this case a medical practitioner, trained nursing personnel or the like, to look into an interior 4 of the device 1 without having to open it for this purpose. In this way it is also possible, for example, to look for leaks or stuck pumps. This can also be done during operation without having to open the device 1. A further advantage of the switchable housing section 3 is that the pane of the housing section 3 can be switched transparent after hot disinfection to accelerate the cooling process. For this purpose, the housing section 3 is permeable to thermal radiation in the transparent state.

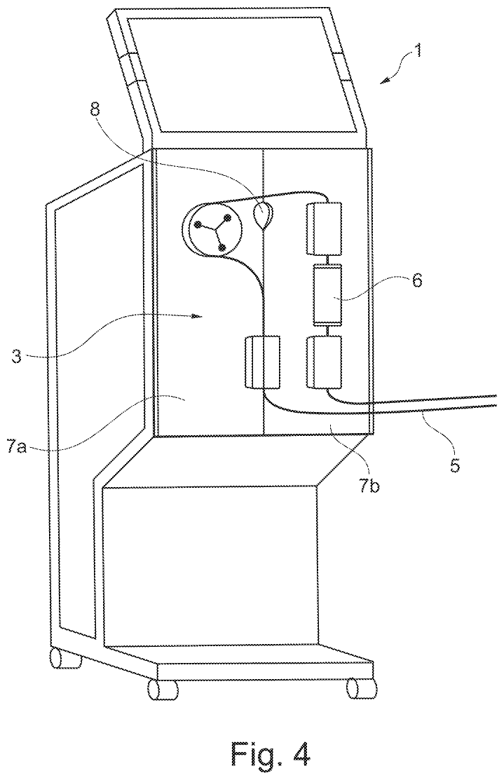

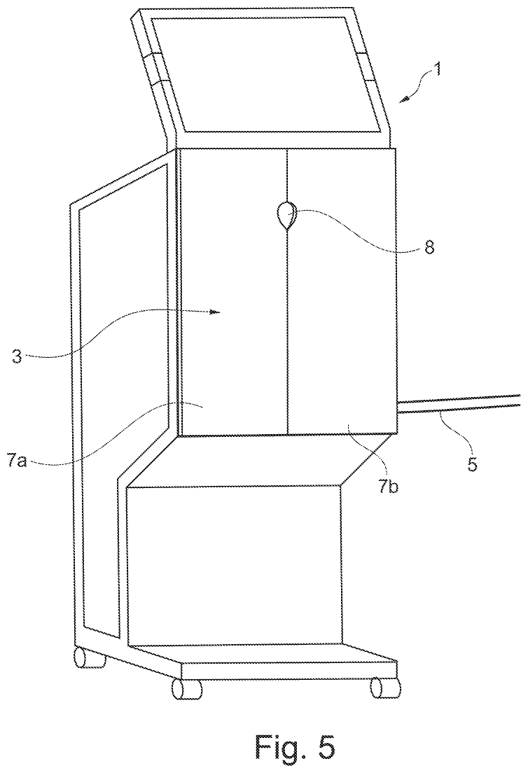

A further configuration example of the medical device 1 according to the invention is shown in FIGS. 4 and 5. Thus, in FIG. 4 the medical device 1 can be seen to which a blood hose system 5 and a dialyzer 6 are attached. The blood hose system 5 and the dialyzer 6 are located behind the housing section 3, which in the present case is in the form of two swing doors 7a and 7b. The swing doors 7a and 7b are designed according to a housing section as shown in FIG. 2, i.e. they each contain at least one liquid crystal foil or something similar and are electrically switchable between a non-transparent initial state and a transparent final state. The swing doors 7a and 7b each have a notch in the upper area, wherein these notches are arranged opposite to each other and together form an opening 8. This opening 8 allows a hand, for example, to reach in and open the swing doors 7a and 7b. Of course, instead of the opening 8 and the notches associated therewith, a handle or another means to be handled can also be used to open the swing doors 7a and 7b. Furthermore, a simple door with an abutment on one side of the housing 2 is also conceivable. This abutment may be in the form of hinges on each longitudinal side of the housing 2.

In the present configuration example, it is essential that the housing 2 can be opened, since the interior of the medical device 1 is/will be equipped—i.e., set up—with single-use items. In the configuration example shown in FIG. 4, the housing section 3 is transparent and allows viewing of the disposable blood hose system 5, the dialyzer 6 and the other common components of the medical device 1.

The medical device 1 shown in FIG. 5 is designed according to the embodiment of the medical device 1 shown in FIG. 4. In FIG. 5, the housing section 3, i.e. the swing doors 7a and 7b, is switched to non-transparent. Thus, no electrical voltage is applied to the foil 3c. The components at the front of the medical device 1 as well as the dialyzer 6 and the majority of the blood hose system 5 are therefore not visible.

This is advantageous in particular if the patient wishes to avoid the sight of blood in the extracorporeal blood circuit. Another advantage is that the blood in the blood hose system 5 behind such a housing section 3 is on the one hand protected from cooling, and on the other hand is not unnecessarily heated, for example by solar radiation. According to another configuration example not explicitly shown, it would also be conceivable to provide only parts of the housing section 3, or respectively of the swing doors 7a and 7b, with an electrically switchable foil 3c, which can be switched to transparent or non-transparent as required. Instead of using swing doors 7a and 7b or pivot doors in general, a retractable cover can alternatively be used as an electrically-switchable housing section 3, which is pulled over the machine front, for example, with the aid of a rail system. A design as a roller shutter is also conceivable if roll-up and/or flexible materials are used for the electrically switchable foil 3c.

Of course, the number of housing sections 3 as well as their shape and size are not limited to the examples shown in FIGS. 1 to 5. Several housing sections 3 can be implemented per medical device 1, which are designed to be electrically switchable. Also only partial sections of housing sections 3 are implementable according to the aforementioned design.

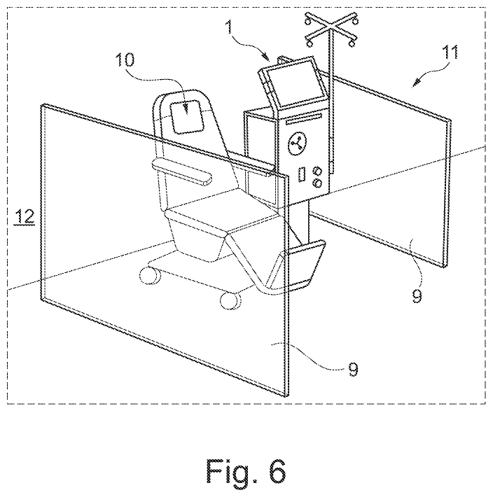

FIG. 6 shows a treatment station 11 for dialysis in schematic form. A treatment couch 10 is provided next to the medical device 1. The treatment station 11 is arranged on a rear building wall 12, in which a ring circuit with several connections (not shown further), if applicable, is laid. From this wall 12, electrically switchable separation walls 9 extend to both sides of the treatment station 11 according to the principle of the housing part 3. These could be switched to non-transparent, for example, if a patient wishes to have some privacy or if there is an emergency and the staff wants to protect the emergency patient from prying eyes of other patients. In addition to a stationary installation of the separation walls 9, it is conceivable that the separation walls 9 are extendable or foldable or can be rolled out or moved in a paravant-like manner and are thus only pulled out and switched non-transparent/transparent when required. In addition to a previously described arrangement of the separation walls 9 extending from the rear wall 12, it is conceivable that the separation walls 9 are a component of the treatment couch 10 and/or of the medical device 1. This would result in a much more mobile application.

A medical device for extracorporeal blood treatment having a housing section that is reversibly variable between two states is disclosed.

1. A medical device for extracorporeal blood treatment, the medical device comprising a housing and an extracorporeal blood hose system arranged at least partially in the housing, wherein the housing has a plurality of housing sections, wherein at least a part of one of the housing sections is connected to an electrical voltage source and is configured to be reversibly changeable between at least two transparency states depending on a currently applied electrical voltage.

2. The medical device according to claim 1, wherein the housing section is non-transparent in a first state.

3. The medical device according to claim 2, wherein, in the first state, an interior of the housing is visually covered.

4. The medical device according to claim 1, wherein the housing section is transparent in a second state.

5. The medical device according to claim 4, wherein in the second state, an interior of the housing is viewable.

6. The medical device according to claim 1, wherein the housing section is switchable exclusively between two states.

7. The medical device according to claim 6, wherein a switching operation activates an electrically switchable foil arranged between two layers of transparent material which together form a pane of said one of the housing sections.

8. The medical device according to claim 7, wherein the foil comprises a polymer liquid crystal film, nano-crystallines or micro-blinds, is electrochromic, or is a suspended particle device.

9. A housing section for a medical device according to claim 1.

10. A method for temporary visualization of an extracorporeal blood hose system of a blood treatment apparatus, comprising the steps of:
accommodating the extracorporeal blood hose system at least partially within a housing of an extracorporeal blood treatment device; and
applying an electric voltage to make a part of a housing section, which is non-transparent in an initial state, transparent by applying an electric voltage, in order to make the extracorporeal blood hose system visible from outside the housing without having to open the housing.

11. A treatment station with a medical device according to claim 1, wherein the treatment station is configured on at least one side with an electrically-switchable separation wall corresponding to the part of the housing section.

12. The medical device according to claim 2, wherein the extracorporeal blood hose system is visually covered in the housing in the first state.

13. The medical device according to claim 4, wherein the extracorporeal blood hose system is viewable in the housing in the second state.
